(19) United States
(12) Patent Application Publication
(10) Pub. No.: US 2014/0296154 A1
(43) Pub. Date: Oct. 2, 2014

(54) METHOD OF PREVENTING DEVELOPMENT OF PSORIATIC LESIONS

(71) Applicants: Brian Poligone, Fairport, NY (US); Sankar Ghosh, New York, NY (US)

(72) Inventors: Brian Poligone, Fairport, NY (US); Sankar Ghosh, New York, NY (US)

(73) Assignees: YALE UNIVERSITY, New Haven, CT (US); UNIVERSITY OF ROCHESTER, Rochester, NY (US)

(21) Appl. No.: 14/115,036

(22) PCT Filed: May 7, 2012
(86) PCT No.: PCT/US12/36807

§ 371 Date: Jun. 16, 2014

Related U.S. Application Data
(60) Provisional application No. 61/483,401, filed on May 6, 2011.

(51) Int. Cl. C07K 14/435; C07K 14/47; A61K 45/06; A61K 38/17

(52) U.S. Cl. CPC C07K 14/435; A61K 38/1709; C07K 14/47; A61K 45/06
USPC 514/18.7; 514/44 R

(57) ABSTRACT

The present invention relates to a method of inhibiting onset of or preventing development of a psoriatic lesion in a patient having psoriasis. The method comprises administering to a patient having psoriasis an effective amount of an agent that inhibits NF-κB activity under conditions effective to inhibit onset of or prevent development of psoriatic lesions. Another aspect of the invention relates to a method of treating an early stage psoriatic lesion on a patient by contacting the early stage psoriatic lesion of a patient with an effective amount of an agent that inhibits NF-κB activity, whereby said contacting inhibits development of the early stage psoriatic lesion. Both transgenic and nontransgenic approaches are contemplated.

This application claims the benefit of U.S. Provisional Patent Application Ser. No. 61/483,401, filed May 6, 2011, which is hereby incorporated by reference in its entirety.

### FIELD OF THE INVENTION

The present invention relates to a method of preventing development of psoriatic lesions or treating psoriatic lesions in a patient having psoriasis.

### BACKGROUND OF THE INVENTION

Psoriasis is a skin disorder that includes the presence of small elevations of the skin that may be characterized as elevated red plaques or pustules on the skin which eventually result in silvery scales. These silvery scales and plaque are the result of accelerated epidermal proliferation, the metabolic activity and proliferation of capillaries in the dermal region, and the invasion of the dermis and epidermis by inflammatory cells. More specifically, the capillaries in the dermal region become tortuous and dilated, and an inflammatory reaction causes the skin to redden.

Psoriasis is thought to be driven primarily by CD4(+) T cells with a T(h)1 and/or T(h)17 phenotype. The severity and course of psoriasis can vary greatly depending on the individual, but in general this chronic skin condition recurs throughout the life of the individual with varying intervals of one month to many years.

The areas affected by psoriasis include scalp, face, body, arms, legs, nails etc. Psoriasis can occur as a few lesions or can be widely distributed over the whole body. Psoriasis can present itself in many forms, such as plaque-type, guttate, inverse and erythrodermic. It often appears between the ages of 15-35, but can develop at any age. In rare cases, it can affect infants. An estimated 2-3% of world's population is affected by psoriasis.

Over the years a wide variety of topical and systemic treatment methods that inhibit the cell division have been developed for psoriasis. In general, these methods have met with limited short term success and are not very well understood. As the disease requires treating the individual intermittently during their lifetime, treatment risk increases with treatment length since common medicaments evidence cumulative long term side effects. Well-known treatments for psoriasis include topical steroid creams and ointments that are administered to psoriatic lesions. Unfortunately many of these drugs produce serious side effects, and in some cases once the drugs are discontinued, the psoriasis recurs with marked exacerbation. The specific topical treatments also include corticosteroids, coal tar, anthralin, vitamin D3 (Dovonex) and Protopic ointment. Systemic medications to treat psoriasis include methotrexate, oral retinoid, cyclosporine, mycophenolate mofetil, sulfasalazine and 6-Thioguanine. Another type of systemic treatment for psoriasis includes biologic drugs such as Amevive, Enbrel, Humira, Raptiva, Stelera and Remicade. On the whole, these prior treatments have proven to be of limited value, and there remains a need for new psoriasis treatments.

Although U.S. Pat. No. 7,538,089 to May et al. describes anti-inflammatory compounds that inhibit binding of NF-κB Essential Modulator (known as “NEMO”) to IκB protein kinase (IKK) and recites their use for the treatment of psoriasis, there is no recognition in this reference that the disclosed compounds are incapable of treating active or advanced psoriasis lesions, as shown in the accompanying Examples. Thus, there remains a need to treating patients with psoriasis to prevent the development of their psoriatic lesions.

The present invention overcomes these and other deficiencies in the art.

### SUMMARY OF THE INVENTION

A first aspect of the present invention relates to a method of inhibiting onset of or preventing development of psoriatic lesions in a patient having psoriasis. The method includes administering to a patient having psoriasis an effective amount of an agent that inhibits NF-κB activity under conditions effective to inhibit onset of or prevent development of psoriatic lesions.

In certain embodiments, the patient to be treated is free of late stage psoriatic lesions and has only early stage psoriatic lesions. In these embodiments, the treatment is effective to prevent or inhibit further development of the psoriatic lesions. In certain embodiments, the patient to be treated is asymptomatic, i.e., free of both late stage psoriatic lesions and early stage psoriatic lesions. In these embodiments, the treatment is effective to inhibit onset of the psoriatic lesions.

A second aspect of the present invention relates to a method of treating a psoriatic lesion on a patient. The method includes contacting an early stage psoriatic lesion of a patient with an effective amount of an agent that inhibits NF-κB activity, whereby said contacting inhibits further development of the psoriatic lesion.

As demonstrated in the accompanying Examples, transgenic keratin 14 (K14)/vascular endothelial growth factor (VEGF) mice develop a psoriasis-like phenotype that is representative of disease in humans. This is a well accepted model of the human condition. Using these K14/VEGF transgenic mice, the Examples demonstrate that treatment of individuals having advanced or late stage psoriatic lesions with an inhibitor of NF-κB activity had no effect on the psoriatic lesions whereas treatment of juvenile individuals prior to onset of psoriatic lesions or having only early stage psoriatic lesions resulted in a significant inhibition of the development psoriatic lesions. The accompanying Examples also offer an explanation for these results, whereby early stage lesions, exhibiting low expression levels of the innate NF-κB inhibitor A20, exhibit NF-κB dependent inflammatory responses while advanced psoriatic lesions exhibit NF-κB independent inflammatory responses. These Examples demonstrate that the timing of intervention is important to the management of psoriasis.

### BRIEF DESCRIPTION OF THE DRAWINGS

FIG. 1 illustrates the canonical NF-κB pathway (figure from Hayden et al., “Shared Principles in NF-kappaB Signaling,” Cell 132(3):344-62 (2008), which is hereby incorporated by reference in its entirety). The present invention targets this pathway with an inhibitor known to disrupt NF-κB signaling.

FIG. 2 illustrates the mechanism of the innate NF-κB inhibitor, A20 (or TNFAIP3) (figure from Vereecke et al., “The Ubiquitin-editing Enzyme A20 (TNFAIP3) Is a Central Regulator of Immunopathology,” Trends in Immunology 30(8):383-91 (2009), which is hereby incorporated by reference in its entirety). A20 is a deubiquitinating enzyme.

FIGS. 3A-C show a mouse model used in screening psoriasis treatments in accordance with the present invention. FIGS. 3A and 3B show that a backcrossed, homozygous K14-VEGF transgenic mouse (“PSX” mouse) develops skin lesions within 8 weeks of birth, which persist into adulthood and have a pathology that is nearly identical to human psoriasis. This is a well accepted model of the human condition. FIGS. 3C and 3D show pathology of the PSX mouse model. “N” indicates neutrophilic abscess, “R” shows elongated Rete Ridges, and arrows indicate dilated capillary vessels.

FIG. 4 is an image of a Western blot illustrating the effect of VEGF-A on A20 expression levels in MCF-7 cells transfected to overexpress A20. Actin control shows that the effects of VEGF-A are specific.

FIG. 5 is an image of a gel showing detection of PCR products following quantitative RT-PCR analysis of A20 and actin mRNA expression levels in wildtype (WT) mice, WT mice that were treated with 10 microliters 12-O-tetradecanoyl phorbol-13-acetate (“TPA”), and young PSX mice with psoriasis (4 weeks).

FIG. 6 is an image of a Western Blot using samples obtained from WT mice (lane 1), WT mice treated with TPA (lane 2), 24-week old PSX mice without and with TPA treatment (lanes 3 and 4), and 4-week old PSX mice without and with TPA treatment (lanes 5 and 6). Anti-A20 antibody at 1:1000 dilution was used as the primary antibody, and a secondary anti-IgG-HRP at 1:10000 was used to develop the blot. Arrow indicates A20 band.

FIG. 7 is a graph illustrating the effect of an NF-κB inhibitor on older PSX mice after they had fully developed psoriatic skin lesions. The NF-κB inhibitor was administered by intraperitoneal injection twice weekly at 50 micrograms per injection. Relative to the untreated control (solid diamond), treatment with the NF-κB inhibitor (solid square) had no change on mean ear thickness in mice with established psoriatic plaques over 8 weeks of treatment.

FIG. 8 is a graph illustrating the effect of an NF-κB inhibitor on young PSX mice that began receiving treatments prior to the development of psoriatic plaques when the ears were still uninvolved. The NF-κB inhibitor was administered by intraperitoneal injection twice weekly at 50 micrograms per injection. Treatment with the NF-κB inhibitor (solid circle) prevented the thickening of the ear and development of phenotypic lesions of psoriasis, as seen in vehicle (DMSO) controls (solid square).

FIG. 9 is an image comparing the appearance of PSX mice representative of the treated cohort and the control cohort from the experiment illustrated in FIG. 8. The treatment quite clearly prevented the thickening of the ear and development of phenotypic lesions of psoriasis as seen in vehicle controls (right).

FIGS. 10A-B are images of skin tissue samples following topical administration of a composition containing FITC-conjugated NBD peptide of SEQ ID NO: 122. In FIG. 10A, peptide is limited to the stratum corneum following administration of the peptide in vaseline ointment; the peptide was not absorbed into the skin. In FIG. 10B, peptide uptake is prevalent among the cells in both the epidermis and dermis following administration of the peptide in DMSO.

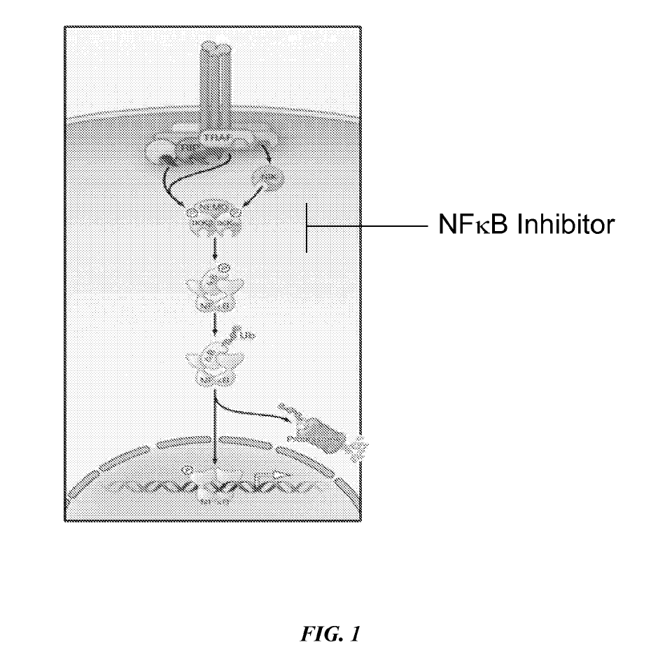

### DETAILED DESCRIPTION OF THE INVENTION

The present invention relates to a method of inhibiting onset of or preventing development of psoriatic lesions in a patient having psoriasis. The method includes administering to a patient having psoriasis an effective amount of an agent that inhibits NF-κB activity under conditions effective to inhibit onset of or prevent development of psoriatic lesions.

The present invention also relates to a method of treating a psoriatic lesion on a patient. The method includes contacting an early stage psoriatic lesion of a patient with an effective amount of an agent that inhibits NF-κB activity, whereby said contacting inhibits further development of the psoriatic lesion.

As used herein, the terms “subject” and “patient” are used interchangeably and include warm-blooded animals, preferably mammals. In a preferred embodiment, the subject is a primate such as a human. In certain embodiments, the subject or patient is characterized by having low levels of A20 expression, which normally acts to inhibit NF-κB activity. Together, NF-κB and A20 maintain inflammatory responses in check, but lower than normal A20 expression levels allow NF-κB to initiate pro-inflammatory responses associated with early stage development of psoriatic lesions.

As used herein, “inhibiting onset of a psoriatic lesion” means that the skin, or a region of skin on the patient, that is susceptible to development of psoriasis is free of any psoriatic lesions. Thus, in certain embodiments, the patient or portions of the patient's skin to be treated is asymptomatic, i.e., free of both late stage psoriatic lesions and early stage psoriatic lesions. In these embodiments, the treatment is effective to inhibit onset of the psoriatic lesions.

As used herein, “preventing development of a psoriatic lesion” means that the skin on the subject has one or more early stage psoriatic lesions, and the treatment of the patient is effective to prevent expansion of those lesions. The term “early stage psoriatic lesion” is intended to encompass lesions that are less than about 25 mm², preferably less than about 20 mm², more preferably less than about 15 mm² or even less than about 10 mm² The early stage psoriatic lesions are also preferably characterized by low expression levels of A20. Thus, in certain embodiments, the patient to be treated is free of late stage psoriatic lesions and has only early stage psoriatic lesions. In these embodiments, the treatment is effective to prevent or delay development of these psoriatic lesions.

In certain embodiments, the agents that inhibit NF-κB activity are administered while NF-κB influences early stages of the inflammatory response associated with psoriatic lesion development. The accompanying examples demonstrate that these agents are ineffective in treating advanced psoriatic lesions, where NF-κB no longer drives the inflammatory response.

Thus, in accordance with the present invention, an effective amount of the agents described herein are administered to a patient so as to prevent formation of new psoriatic lesions or to prevent or inhibit expansion of early stage psoriatic lesions.

In the various embodiments of the present invention, an agent that inhibits NF-κB activity is used. One exemplary class of NF-κB inhibitors is based, at least in part, on the identification of the NEMO binding domain (NBD) on IκB kinase-α (IKKα) and on IκB kinase-β (IKKβ) as an agent that is effective in preventing development of early stage psoriatic lesions. More specifically, these agents are effective for preventing development of lesions in the first place or for inhibiting development of early stage lesions where NF-κB remains active in influencing an inflammatory response.

Without intending to be limited by mechanism, it is believed that these agents act by blocking the interaction of NEMO with an IKK (e.g., IKKβ or IKKα) at the NEMO binding domain (NBD), thereby inhibiting phosphorylation, degradation and subsequent dissociation of IκB from NF-κB. This inhibition results in blockade of NF-κB activation associated with pro-inflammatory responses caused during early stages of psoriatic lesion formation.

As used herein, the term “NEMO Binding Domain” or “NBD” includes any domain capable of binding to NEMO at the region where NEMO usually interacts with an IKK (e.g., IKKα or IKKβ). NEMO binding domains include, for example, the α2-region (residues 737-742) of wild-type IKKβ, or the corresponding six amino acid sequence of wild-type IKKα (residues 738-743) which are involved in interaction with NEMO. The nucleic acid sequence and the corresponding amino acid sequence of the wild-type IKKβ NBD are provided in GenBank Accession No. AR067807; nucleotides 2203-2235; see also U.S. Pat. No. 7,534,858 to May et al., U.S. Pat. No. 7,812,118 to May et al., U.S. Pat. No. 7,538,089 to May et al., and U.S. Pat. No. 7,872,094 to May et al., and PCT International Patent Publication Nos. WO 01/83554 to May et al., and WO 01/83547 to May et al., each of which is hereby incorporated by reference in its entirety.

These agents are capable of down-regulating NEMO. Down-regulation is defined herein as a decrease in activation, function or synthesis of NEMO, its ligands or activators. It is further defined to include an increase in the degradation of the NEMO gene, its protein product, ligands or activators. Down-regulation may be achieved in a number of ways, for example, by destabilizing the binding of NEMO to an IKK (e.g., IKKβ or IKKα); or by blocking the phosphorylation of IκB and causing the subsequent degradation of this protein.

Phosphorylation of IκB by IKKβ results in ubiquitination and degradation of IκB and subsequent dissociation of IκB, allowing for nuclear translocation of NF-κB, leading to up-regulation of genes critical to the inflammatory response. The agents that inhibit that inhibit NF-κB activity may therefore be used to down-regulate NF-κB function. Down-regulation of NF-κB may also be accomplished by using polyclonal or monoclonal antibodies or fragments thereof directed against a NBD or NEMO itself This invention further includes the use of small molecules having the three-dimensional structure necessary to bind with sufficient affinity to a NBD or NEMO itself to, e.g., block NEMO interactions with IKKβ. IKKβ blockade resulting in decreased degradation of IκB and decreased activation of NF-κB make these small molecules useful as therapeutic agents in treating or preventing inflammation. This invention also includes the use of small molecules that act directly upon IKK to inhibit its activity.

In one embodiment, the present invention provides an agent that has the formula

Xₐ—X_{b},

where Xₐ is a membrane translocation domain comprising up to about 20 or 25 amino acid residues, more preferably from 6 to 15 amino acid residues; and X_{b} is a NEMO binding sequence. The agent can optionally include a modifying group at the N-terminus, the C-terminus or both.

X_{b} is a NEMO binding sequence comprising from 6 to 9 amino acid residues. In one embodiment, X_{b} consists of the following structure

(Y)ₙ—X₁—X₂—X₃—X₄—X₅—X_{B}-(A)ₘ

where n and m are each, independently, 0 or 1 and A and Y each comprises from 1 to about 3 amino acid residues. When n is 1, Y is, preferably the sequence TA. When m is 1, A is preferably the sequence QTE. X₁ is L, A, I or nor-leucine (Nle); X₂ is D, E, N, Q, homoserine (Hser) or 2-ketopropylalanine (2-ketopropy-A); X₃ is W, F Y, 4-biphenyl-alanine (Bpa), homophenylalanine (Hphe), 2-Naphthylalanine (2-Nal), 1-Naphthylalanine (1-Nal), or cycloxexyl-alanine (Cha); X₄ is S, A, E, L, T, nor-leucine (Nle), or homoserine (Hser); X₅ is W, H, homophenylalanine (Hphe), 2-Naphthylalanine (2-Nal), 1-Naphthylalanine (1-Nal), O-benzyl serine (SeroBn), or 3-Pyridylalanine (3-Pal); and X₆ is L, A, I, or nor-leucine (Nle).

Preferably, X_{b} is a sequence selected from among TALDWSWLQTE (SEQ ID NO: 1); LDWSWLQTE (SEQ ID NO: 2); TALDWSWL (SEQ ID NO: 3); ALDWSWLQTE (SEQ ID NO: 4); LDWSWLQTE (SEQ ID NO: 5); LDWSWL (SEQ ID NO: 6); TALDWSWLQT (SEQ ID NO: 7); TALDWSWLQ (SEQ ID NO: 8); ALDWSWLQT (SEQ ID NO: 9); LDWSWLQ (SEQ ID NO: 10); LDWSWLQT (SEQ ID NO: 11); ADWSWL (SEQ ID NO: 12); LDWSWA (SEQ ID NO: 13); ADWSWA (SEQ ID NO: 14); LDFSWL (SEQ ID NO: 15); LDYSWL (SEQ ID NO: 16); LDWAWL (SEQ ID NO: 17); LDWEWL (SEQ ID NO: 18); TAADWSWLQTE (SEQ ID NO: 19); ADWSWLQTE (SEQ ID NO: 20); TAADWSWL (SEQ ID NO: 21); AADWSWLQTE (SEQ ID NO: 22); ADWSWLQTE (SEQ ID NO: 23); ADWSWL (SEQ ID NO: 24); TAADWSWLQT (SEQ ID NO: 25); TAADWSWLQ (SEQ ID NO: 26); AADWSWLQT (SEQ ID NO: 27); ADWSWLQ (SEQ ID NO: 28); ADWSWLQT (SEQ ID NO: 29); ALDWSWAQTE (SEQ ID NO: 30); LDWSWAQTE (SEQ ID NO: 31); TALDWSWA (SEQ ID NO: 32); ALDWSWAQTE (SEQ ID NO: 33); LDWSWAQTE (SEQ ID NO: 34); LDWSWA (SEQ ID NO: 35); TALDWSWAQT (SEQ ID NO: 36); TALDWSWAQ (SEQ ID NO: 37); ALDWSWAQT (SEQ ID NO: 38); LDWSWAQ (SEQ ID NO: 39); LDWSWAQT (SEQ ID NO: 40); TAADWSWAQTE (SEQ ID NO: 41); ADWSWAQTE (SEQ ID NO: 42); TAADWSWA (SEQ ID NO: 43); AADWSWAQTE (SEQ ID NO: 44); ADWSWAQTE (SEQ ID NO: 45); ADWSWA (SEQ ID NO: 46); TAADWSWAQT (SEQ ID NO: 47; TAADWSWAQ (SEQ ID NO: 48); AADWSWAQT (SEQ ID NO: 49); ADWSWAQ (SEQ ID NO: 50); ADWSWAQT (SEQ ID NO: 51); TALDFSWLQTE (SEQ ID NO: 52); LDFSWLQTE (SEQ ID NO: 53); TALDFSWL (SEQ ID NO: 54); ALDFSWLQTE (SEQ ID NO: 55); LDFSWLQTE (SEQ ID NO: 56); LDFSWL (SEQ ID NO: 57); TALDFSWLQT (SEQ ID NO: 58); TALDFSWLQ (SEQ ID NO: 59); ALDFSWLQT (SEQ ID NO: 60); LDFSWLQ (SEQ ID NO: 61); LDFSWLQT (SEQ ID NO: 62); TALDYSWLQTE (SEQ ID NO: 63); LDYSWLQTE (SEQ ID NO: 64); TALDYSWL (SEQ ID NO: 65); ALDYSWLQTE (SEQ ID NO: 66); LDYSWLQTE (SEQ ID NO: 67); LDYSWL (SEQ ID NO: 68); TALDYSWLQT (SEQ ID NO: 69); TALDYSWLQ (SEQ ID NO: 70); ALDYSWLQT (SEQ ID NO: 71); LDYSWLQ (SEQ ID NO: 72); LDYSWLQT (SEQ ID NO: 73); TALDWAWLQTE (SEQ ID NO: 74); LDWAWLQTE (SEQ ID NO: 75); TALDWAWL (SEQ ID NO: 76); ALDWAWLQTE (SEQ ID NO: 77); LDWAWLQTE (SEQ ID NO: 78); LDWAWL (SEQ ID NO: 79); TALDWAWLQT (SEQ ID NO: 80); TALDWAWLQ (SEQ ID NO: 81); ALDWAWLQT (SEQ ID NO: 82); LDWAWLQ (SEQ ID NO: 83); LDWAWLQT (SEQ ID NO: 84); TALDWEWLQTE (SEQ ID NO: 85); LDWEWLQTE (SEQ ID NO: 86); TALDWEWL (SEQ ID NO: 87); ALDWEWLQTE (SEQ ID NO: 88); LDWEWLQTE (SEQ ID NO: 89); LDWEWL (SEQ ID NO: 90); TALDWEWLQT (SEQ ID NO: 91); TALDWEWLQ (SEQ ID NO: 92); ALDWEWLQT (SEQ ID NO: 93); LDWEWLQ (SEQ ID NO: 94); and LDWEWLQT (SEQ ID NO: 95).

Xₐ is a membrane transduction domain containing up to 20 to 25 amino acid residues, preferably containing or consisting of 6-15 amino acid residues, more preferably 6-12, or 6-10 amino acid residues. Preferably, Xₐ is a membrane translocation domain which comprises at least five basic amino acid residues, preferably at least five residues independently selected from L-arginine, D-arginine, L-lysine and D-lysine. Suitable membrane transduction domains include those disclosed herein. The translocation peptide of the present invention may be the third helix of antennapedia homeodomain protein, HIV-1 protein Tat, or a membrane translocation domain peptide as disclosed in Derossi et al., “The Third Helix of the Antennapedia Homeodomain Translocates Through Biological Membranes,” J. Biol. Chem. 269:10444-10450 (1994); Lindgren et al., “Cell-Penetrating Peptides,” Trends Pharmacol. Sci. 21:99-103 (2000); Ho et al., “Synthetic Protein Transduction Domains: Enhanced Transduction Potential In Vitro and In Vivo,” Cancer Research 61:474-477 (2001); U.S. Pat. No. 5,888,762 to Joliot et al.; U.S. Pat. No. 6,015,787 to Potter et al.; U.S. Pat. No. 5,846,743 to Janmey et al.; U.S. Pat. No. 5,747,641 to Frankel et al.; U.S. Pat. No. 5,804,604 to Frankel et al.; PCT Publ. WO 98/52614 to Rothbard et al.; PCT Publ. WO 00/29427 to Fischer et al.; and PCT Publ. WO 99/29721 to Dowdy, S., all of which are hereby incorporated by reference in their entirety.

In one embodiment, Xₐ is selected from among the amino acid sequences RRMKWKK (SEQ ID NO: 96); YGRKKRRQRRR (SEQ ID NO: 97); ygrkkrrqrrr (SEQ ID NO: 98); YARKARRQARR (SEQ ID NO: 99); yarkarrqarr (SEQ ID NO: 100); YARAARRAARR (SEQ ID NO: 101); yaraarraarr (SEQ ID NO: 102); rrmkwkk (SEQ ID NO: 103); (R)_{y} and (r)_{y}, where y is 6 to 11; poly-L-Arg or poly-D-Arg comprising 6 to 11 Arg residues. Lower case letters indicate D-amino acid residues and upper case letters indicate L-amino acid residues.

Examples of suitable peptides Xₐ—X_{b} include those having the following sequences: RRMKWKKTALDWSWLQTE (SEQ ID NO: 104); rrmkwkkTALDWSWLQTE (SEQ ID NO: 105); YGRKKRRQRRRTALDWSWLQTE (SEQ ID NO: 106); ygrkkrrqrrrTALDWSWLQTE (SEQ ID NO: 107); rrrrrrrTALDWSWLQTE (SEQ ID NO: 108); RRRRRRRTALDWSWLQTE (SEQ ID NO: 109); YARKARRQARRTALDWSWLQTE (SEQ ID NO: 110); yarkarrqarrTALDWSWLQTE (SEQ ID NO: 111); YARAARRAARRTALDWSWLQTE (SEQ ID NO: 112); yaraarraarrTALDWSWLQTE (SEQ ID NO: 113); YGRKKRRQRRRLDWSWL (SEQ ID NO: 114); ygrkkrrqrrrLDWSWL (SEQ ID NO: 115); RRMKWKKLDWSWL (SEQ ID NO: 116); rrmnkwkkLDWSWL (SEQ ID NO: 117); rrrrrrrLDWSWL (SEQ ID NO: 118); YARAARRAARRLDWSWL (SEQ ID NO: 119); yaraarraarrLDWSWL (SEQ ID NO: 120); RRRRRRRLDWSWL (SEQ ID NO: 121); and drqikiwfqnrrmkwkkTALDWSWLQTE (SEQ ID NO: 122).

These agents can optionally include modifying groups attached to the C-terminus, the N-terminus or both. For example, suitable modifying groups which can be attached to the C-terminus include substituted and unsubstituted amino groups, for example, —NH₂, —NH(alkyl) and —N(alkyl)₂ groups; and alkoxy groups, such as linear, branched or cyclic C₁-C₆-alkoxy groups. A preferred C-terminal modifying group is the —NH₂ group. Suitable modifying groups which can be attached to the N-terminus include acyl groups, such as the acetyl group; and alkyl groups, preferably C₁-C₆-alkyl groups, more preferably methyl. Any of the peptides listed in the preceding paragraph can be modified in this manner.

In these agents, the membrane translocation domain, Xₐ, may be present at the amino-terminus of the compound and the NEMO binding sequence, X_{b}, may be present at the carboxyl-terminus of the compound (Xₐ—X_{b}). Alternatively, in these agents the membrane translocation domain, Xₐ, may be present at the carboxyl-terminus of the compound and the NEMO binding sequence, X_{b}, may be present at the amino-terminus of the compound (X_{b}—Xₐ).

Another class of agents that inhibits NF-κB activity include those that directly inhibit IKK. Use of any such IKK inhibitor is contemplated herein. Exemplary IKK inhibitors include, without limitation, PS1145, PS341, thalidomide, bortezomib, herbimycin A, sodium salicylate, a retinoid-related compound, a cyclopentenone prostaglandin, and vinpocetine. See U.S. Patent Publ. No. 2010/0221340 to Yan et al.; U.S. Patent Publ. No. 2004/0166095 to Blazar et al., and U.S. Pat. No. 7,803,758 to Khoshnan et al., each of which is hereby incorporated by reference in its entirety. Additional exemplary IKK inhibitors include, without limitation, the compounds disclosed in PCT Applications WO 2002/046171 (anilinopyrimidine derivatives), WO 2004/022553 (indole or benzimidazole derivatives), and WO 2002/044153 (4-aryl pyridine derivatives); Burke et al., J. Biol Chem. 278:1450-1456 (2003) (BMS-345541, which is 4(2′-aminoethyl)amino-1,8-dimethylimidazo(1,2-a)quinoxaline); Coghlan et al., Inflam. Res. 52:2-5 (2003); Kishore et al., J. Biol. Chem. 278(35):32861-71 (2003) (SC-514, or 4-amino-[2,3′-bithiophene]-5-carboxamide); and Podolin et al., J. Pharmacol. Exp. Ther. 312: 373-381 (2005) (TPCA-1, or 2-[(aminocarbonyl)amino]-5-(4-fluorophenyl)-3-thiophenecarboxamide), each of which is hereby incorporated by reference in its entirety.

The various agents that inhibits NF-κB activity may be used to modulate inflammation so as to prevent development of psoriatic lesions. In accordance with the present invention, an effective amount of the agents described herein is administered to a patient so as to prevent formation of new psoriatic lesions or to inhibit or delay expansion of early stage psoriatic lesions as defined above. In accordance with the present invention, the agents are administered while NF-κB influences early stages of the inflammatory response associated with psoriatic lesion development.

As used herein, the term “administering” to a subject includes dispensing, delivering or applying the agents described above, e.g., in a pharmaceutical formulation to a subject by any suitable route for delivery of the agents to the desired location in the subject, including delivery by either the parenteral route, intramuscular injection, subcutaneous/intradermal injection, intravenous injection, transdermal delivery and administration by the rectal, colonic, vaginal, intranasal or respiratory tract route (e.g., by inhalation).

As used herein, the term “effective amount” includes an amount effective, at dosages and for periods of time necessary, to achieve the desired result, e.g., sufficient to prevent development of psoriatic lesions or inhibit or delay progression of the psoriatic lesion in a subject. An effective amount of the agents, as defined herein may vary according to factors such as the age and weight of the subject, and the ability of the agent to elicit a desired response in the subject. Dosage regimens may be adjusted to provide the optimum therapeutic response. An effective amount is also one in which any toxic or detrimental effects (e.g., side effects) of the agent are outweighed by the therapeutically beneficial effects.

A therapeutically effective amount of these agents (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an agent can include a single treatment or, preferably, can include a series of treatments.

In one example, a subject is treated with an agent that inhibits NF-κB activity in the range of between about 0.1 to 20 mg/kg body weight, once or more daily, or once or more weekly, for between about 1 to 10 weeks, preferably between 2 to 8 weeks, more preferably between about 3 to 7 weeks, and even more preferably for about 4, 5, or 6 weeks. When administered systemically, an amount between 0.01 and 100 mg per kg body weight per day, but preferably about 0.1 to 10 mg per kg, will effect a therapeutic result in most instances. It will also be appreciated that the effective dosage of the agent used for treatment may increase or decrease over the course of a particular treatment.

The agents that inhibit NF-κB activity can be provided alone, or in combination with other agents that modulate a particular pathological process. For example, the agents that inhibit NF-κB activity can be administered in combination with other known anti-inflammatory agents. Known anti-inflammatory agents that may be used in the methods of the invention can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell, N.J., Medical Economics Co., which are hereby incorporated by reference in their entirety. The agents that inhibit NF-κB activity and the additional anti-inflammatory agents may be administered to the subject in the same pharmaceutical composition or in different pharmaceutical compositions (at the same time or at different times).

In certain embodiments of the present invention, one or more additional therapeutic agents is selected from the group of corticosteroids, TNF-α inhibitors, vitamin D analogs, retinoids, calcineurin inhibitors, phototherapy, methotrexate, cyclosporine, hydroxyurea, and thioguanine.

Exemplary corticosteroids of the present invention include, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumetholone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and respective pharmaceutically acceptable derivatives thereof.

Exemplary TNF-α inhibitors include, but are not limited to, metalloproteinase (MMP) inhibitors (excluding methylprednisolone), tetracyclines, chemically modified tetracyclines, quinolones, corticosteroids, thalidomide, lazaroids, pentoxifylline, hydroxamic acid derivatives, carbocyclic acids, minocyclines, napthopyrans, soluble cytokine receptors, monoclonal antibodies towards TNF-α, amrinone, pimobendan, vesnarinone, phosphodiesterase inhibitors, lactoferrin and lactoferrin derived analogous, and melatonin in the form of bases or addition salts together with a pharmaceutically acceptable carrier.

Exemplary vitamin D analogs include, but are not limited to, 1α-25 vitamin D compounds, 1α-dihydroxyvitamin D₃ and vitamin D₂ compounds, vitamin D₂ and vitamin D₃ derivatives such as cholecalciferol, calcifediol, calcitriol, calcipotriol, ergosterol, ergocalciferol, dihydrotachysterol, 1,25-dihydroxyergocalciferol, 25-hydroxydihydrotachysterol, and the vitamin D analogs disclosed in U.S. Pat. No. 4,866,048 to Calverley et al., U.S. Pat. No. 5,716,946 to Deluca et al., U.S. Pat. No. 4,310,511 to Holick, M., U.S. Pat. No. 4,634,692 to Partridge et al., U.S. Pat. No. 4,719,205 to Deluca et al., U.S. Pat. No. 4,410,515 to Holick et al., U.S. Pat. No. 4,521,410 to Holick et al., and U.S. Pat. No. 4,230,701 to Holick et al., which are hereby incorporated by reference in their entirety. Likewise, Prosser et al., “Vitamin D Analogs,” Curr. Med. Chem.—Imm., Endooc. & Metab. Agents 1:217-234 (2001), which is incorporated by reference in its entirety, discloses useful vitamin D analogs.

Exemplary retinoids of the present invention include, but are not limited to, retinal, retinol, retinoic acid, retinyl acetate, retinyl palmitate, retinyl propionate, isotretinoin, synthetic retinoid mimics, and tretinoin. Naturally occurring retinoids suitable for use in the present invention include naturally occurring retinoids such as vitamin A (retinol), vitamin A aldehyde (retinal), vitamin A acid (retinoic acid) and their synthetic and natural congeners. Synthetically prepared retinoids suitable for the present invention include those described in U.S. Pat. No. 5,234,926 to Chandraratna, and U.S. Pat. No. 4,326,055 to Loeliger, which are hereby incorporated by reference in their entirety.

Examples of calcineurin inhibitors used in the present invention include, but are not limited to, Tacrolimus (Prograf®, FK506), FK520, cyclosporin (Neoral®), cyclosporin A, and ISA _{TX}247.

The present invention also includes pharmaceutical compositions comprising the agents together with a pharmaceutically acceptable carrier. Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Gennaro et al., (1995) Remington's Pharmaceutical Sciences, Mack Publishing Company, which is hereby incorporated by reference in its entirety. In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil or synthetic fatty acid esters, for example, ethyl oleate or triglycerides. Aqueous injection suspensions may contain substances which increase the viscosity of the suspension include, for example, sodium carboxymethyl cellulose, sorbitol, and dextran. Optionally, the suspension may also contain stabilizers. Liposomes can also be used to encapsulate the agent for delivery into the cell.

The pharmaceutical formulation for systemic administration according to the invention may be formulated for parenteral or topical administration. Indeed, both types of formulations may be used simultaneously to achieve systemic administration of the active ingredient.

The agents that inhibit NF-κB activity can also be incorporated into pharmaceutical compositions which allow for the sustained delivery of the anti-inflammatory compounds to a subject for a period of at least several weeks to a month or more. Such formulations are described in U.S. Pat. No. 5,968,895 to Gefter et al. and U.S. Pat. No. 6,180,608 B1 to Gefter et al., the contents of each of which are incorporated by reference in their entirety.

The agents used in the methods of treatment described herein may be administered systemically or topically, depending on such considerations as the condition to be treated, need for site-specific treatment, quantity of drug to be administered and similar considerations.

Topical administration may be used in certain embodiments. Any common topical formulation such as a solution, emulsion, suspension, gel, ointment or salve and the like may be employed. Preparations of such topical formulations are well described in the art of pharmaceutical formulations as exemplified, for example, by Remington's Pharmaceutical Sciences, which is hereby incorporated by reference in its entirety. For topical application, these compounds could also be administered as a powder or spray, particularly in aerosol form.

The agents may be administered in pharmaceutical compositions adapted for systemic administration. For intravenous, intraperitoneal or intra-lesional administration, the agents will be prepared as a solution or suspension capable of being administered by injection. In certain cases, it may be useful to formulate these compounds in suppository form or as an extended release formulation for deposit under the skin or intramuscular injection. In a preferred embodiment, the agents may be administered by inhalation. For inhalation therapy the compound may be in a solution useful for administration by metered dose inhalers or in a form suitable for a dry powder inhaler.

In practicing the methods of this invention, the compounds of this invention may be used alone or in combination, or in combination with other therapeutic or diagnostic agents. In certain preferred embodiments, the compounds of this invention may be co-administered along with other compounds typically prescribed for these conditions according to generally accepted medical practice. The compounds of this invention can be utilized in vivo, ordinarily in mammals, preferably in humans. The administration of the agents of the present invention may be carried out after beginning a course of administering, after ending a course of administering, or concurrently with the administering of the one or more additional therapeutic agents.

In still another embodiment, the anti-inflammatory compounds of the invention may be coupled to chemical moieties, including proteins that alter the functions or regulation of target proteins for therapeutic benefit. These proteins may include in combination other inhibitors of cytokines and growth factors that may offer additional therapeutic benefit in the treatment of inflammatory disorders. In addition, the anti-inflammatory compounds of the invention may also be conjugated through phosphorylation to biotinylate, thioate, acetylate, iodinate using any of the cross-linking reagents well known in the art.

In addition to the administration of inhibitors of NF-κB, the present invention also contemplates the prevention of psoriasis lesion development through the use of transgenic expression of the innate NF-κB inhibitor A20. This can be achieved by cloning the A20 cDNA under control of a weakly constitutive promoter or a tissue specific promoter such as K14 or K5 (Li et al., “Targeted Cardiac Overexpression of A20 Improves Left Ventricular Performance and Reduces Compensatory Hypertrophy After Myocardial Infarction,” Circulation 115:1885-1894 (2007); Xia et al., “Transgenic delivery of VEGF to Mouse Skin Leads to an Inflammatory Condition Resembling Human Psoriasis,” Blood 102(1):161-168 (2003); Wang et al., “Transgenic Studies with a Keratin Promoter-driven Growth Hormone Transgene: Prospects for Gene Therapy,” Proc. Nat'l Acad. Sci. USA 94:219-226 (1997), each of which is hereby incorporated by reference in its entirety). Such a transgene can be introduced into epithelial cells of the skin via infective transformation vectors as well as using noninfective approaches such as electroporation or transdermal delivery vehicles with mediated uptake. For example, U.S. Patent Publ. 20060084938 to Zhang et al., which is hereby incorporated by reference in its entirety, describes the delivery of naked DNA to skin by non-invasive in vivo electroporation. These and other procedures can be use to overexpress A20 in skin that is susceptible to psoriatic lesion development.

Because A20 can be transgenically expressed to inhibit NF-κB activity in treating/preventing psoriasis in accordance with the present invention, it is also believed that A20 overexpression under control of constitutive or other tissue-specific promoters also can be used to treat other NF-κB-mediated inflammatory conditions. Exemplary NF-κB-mediated inflammatory conditions that can be treated with A20 gene therapy include, without limitation, asthma, psoriasis, rheumatoid arthritis, osteoarthritis, psoriatic arthritis, inflammatory bowel disease (Crohn's disease, ulcerative colitis), sepsis, vasculitis, and bursitis; autoimmune diseases such as Lupus, Polymyalgia, Rheumatica, Scleroderma, Wegener's granulomatosis, temporal arteritis, cryoglobulinemia, and multiple sclerosis; transplant rejection; osteoporosis; cancer, including solid tumors (e.g., lung, CNS, colon, kidney, and pancreas); Alzheimer's disease; atherosclerosis; viral (e.g., HIV or influenza) infections; chronic viral (e.g., Epstein-Barr, cytomegalovirus, herpes simplex virus) infection; and ataxia telangiectasia.

To achieve transgene administration of an A20-encoding transgene to tissues other than skin, alternative delivery vehicles can be utilized, including infective transformation vectors as well as nanoparticle delivery vehicles, liposomal delivery vehicles, etc.

A variety of nanoparticle delivery vehicles are known in the art and are suitable for transgene delivery (see e.g., van Vlerken et al., “Multi-functional Polymeric Nanoparticles for Tumour-Targeted Drug Delivery,” Expert Opin. Drug Deliv. 3(2):205-216 (2006), which is hereby incorporated by reference in its entirety). Suitable nanoparticles include, without limitation, poly(beta-amino esters) (Sawicki et al., “Nanoparticle Delivery of Suicide DNA for Epithelial Ovarian Cancer Cell Therapy,” Adv. Exp. Med. Biol. 622:209-219 (2008), which is hereby incorporated by reference in its entirety), and polyethylenimine-alt-poly(ethylene glycol) copolymers (Park et al., “Degradable Polyethylenimine-alt-Poly(ethylene glycol) Copolymers As Novel Gene Carriers,” J. Control Release 105(3):367-80 (2005) and Park et al., “Intratumoral Administration of Anti-KITENIN shRNA-Loaded PEI-alt-PEG Nanoparticles Suppressed Colon Carcinoma Established Subcutaneously in Mice,” J Nanosci. Nanotechnology 10(5):3280-3 (2010), which are hereby incorporated by reference in their entirety). Other nanoparticle delivery vehicles suitable for use in the present invention include microcapsule nanotube devices disclosed in U.S. Patent Publication No. 2010/0215724 to Prakash et al., which is hereby incorporated by reference in its entirety.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. Several advantages of liposomes include: their biocompatibility and biodegradability, incorporation of a wide range of water and lipid soluble drugs; and they afford protection to encapsulated contents from metabolism and degradation. Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active transgene may act.

Methods for preparing liposomes for use in the present invention include those disclosed in Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., which are hereby incorporated by reference in their entirety. The liposome and nanoparticle delivery systems can be made to accumulate at a target organ, tissue, or cell via active targeting (e.g., by incorporating an antibody or other ligand on the surface of the delivery vehicle).

In another embodiment, the delivery vehicle is a viral vector. Viral vectors are particularly suitable for the delivery of a transgene. Suitable gene therapy vectors include, without limitation, adenoviral vectors, adeno-associated viral vectors, retroviral vectors, lentiviral vectors, and herpes viral vectors.

Adenoviral viral vector delivery vehicles can be readily prepared and utilized as described in Berkner, “Development of Adenovirus Vectors for the Expression of Heterologous Genes,” Biotechniques 6:616-627 (1988) and Rosenfeld et al., “Adenovirus-Mediated Transfer of a Recombinant Alpha 1-Antitrypsin Gene to the Lung Epithelium In Vivo,” Science 252:431-434 (1991), WO 93/07283 to Curiel et al., WO 93/06223 to Perricaudet et al., and WO 93/07282 to Curiel et al., which are hereby incorporated by reference in their entirety.

Adeno-associated viral delivery vehicles can be constructed and used to deliver a transgene as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-Aβ Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); and Zhou et al., “Adeno-associated Virus 2-Mediated Transduction and Erythroid Cell-Specific Expression of a Human Beta-Globin Gene,” Gene Ther. 3:223-229 (1996), which are hereby incorporated by reference in their entirety. In vivo use of these vehicles is described in Flotte et al., “Stable in Vivo Expression of the Cystic Fibrosis Transmembrane Conductance Regulator With an Adeno-Associated Virus Vector,” Proc. Nat'l. Acad. Sci. 90:10613-10617 (1993) and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Retroviral vectors which have been modified to form infective transformation systems can also be used to deliver a nucleic acid molecule to a target cell. One such type of retroviral vector is disclosed in U.S. Pat. No. 5,849,586 to Kriegler et al., which is hereby incorporated by reference. Other nucleic acid delivery vehicles suitable for use in the present invention include those disclosed in U.S. Patent Publication No. 20070219118 to Lu et al., which is hereby incorporated by reference in its entirety.

Regardless of the type of infective transformation system employed, it should be targeted for delivery of the nucleic acid to the desired cell type. For example, for delivery into a cluster of cells or specific tissue, a high titer of the infective transformation system can be injected directly within the site of those cells or tissue so as to enhance the likelihood of cell infection. The infected cells will then express the transgene and produce A20. As noted above, the expression system can further contain a promoter to control or regulate the strength and specificity of expression of the A20-encoding transgene in a target tissue or cell.

Such administration can be carried out systemically or via direct or local administration to the site where the inflammatory condition is to be treated. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterialy, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art.

### EXAMPLES

The following examples are provided to illustrate embodiments of the present invention but are by no means intended to limit its scope.

### Example 1

### K14/VEGF Mouse Model Replicates Human Psoriasis

Transgenic mice overexpressing VEGF under the Keratin 14 (K14) promoter, which targets gene expression to the basal cells of stratified squamous epithelia, develop an inflammatory skin condition with many of the pathobiological features of human psoriasis. It has been previously reported by Xia et al., “Transgenic Delivery of VEGF to Mouse Skin Leads to an Inflammatory Condition Resembling Human Psoriasis, Blood 102(1):161-168 (2003), which is hereby incorporated by reference in its entirety, that chronic VEGF expression in the skin results in a profound inflammatory condition with many of the cellular and molecular hallmarks of human psoriasis, including hyperplastic and inflamed dermal blood vessels, epidermal thickening with aberrant keratinocyte differentiation, and characteristic inflammatory infiltrates. This is a well accepted model of the human condition.

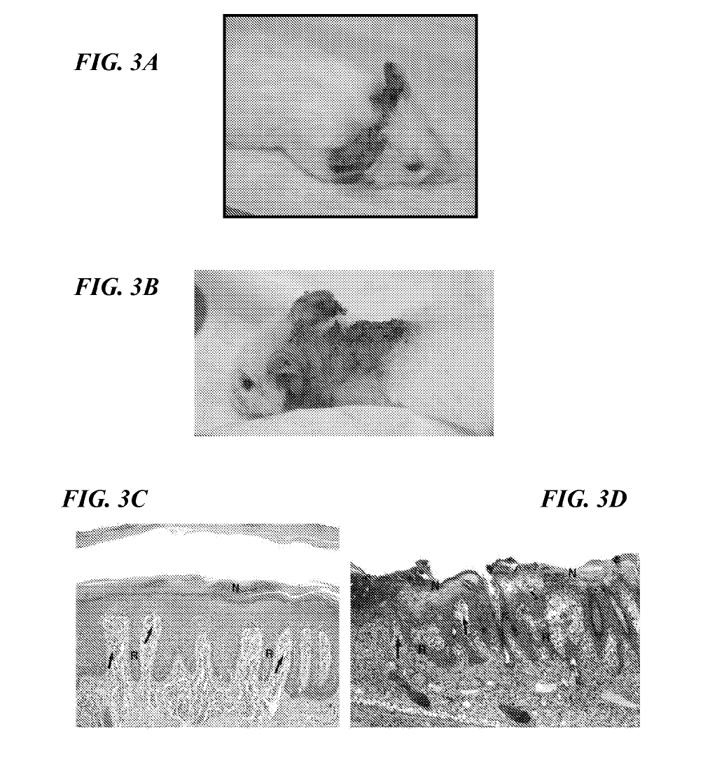

This K14-VEGF mouse was backcrossed to homozygosity for the transgene(s), generating a mouse model that develops psoriasiform lesions on the ears spontaneously and after trauma. These are referred to as PSX mice. Although this model does not develop arthritis, it recapitulates many features of cutaneous plaque psoriasis. FIGS. 3A-D illustrate the PSX transgenic mouse model used in screening psoriasis treatments in accordance with the present invention. FIGS. 3A and 3B show that this mouse develops skin lesions within 8 weeks of birth, which persist into adulthood and have a pathology that is nearly identical to human psoriasis. FIGS. 3C and 3D show pathology of the mouse model. “N” indicates neutrophilic abscess, “R” shows elongated Rete Ridges, and arrows indicate dilated capillary vessels.

### Example 2

### Overexpressed VEGF Downregulates A20

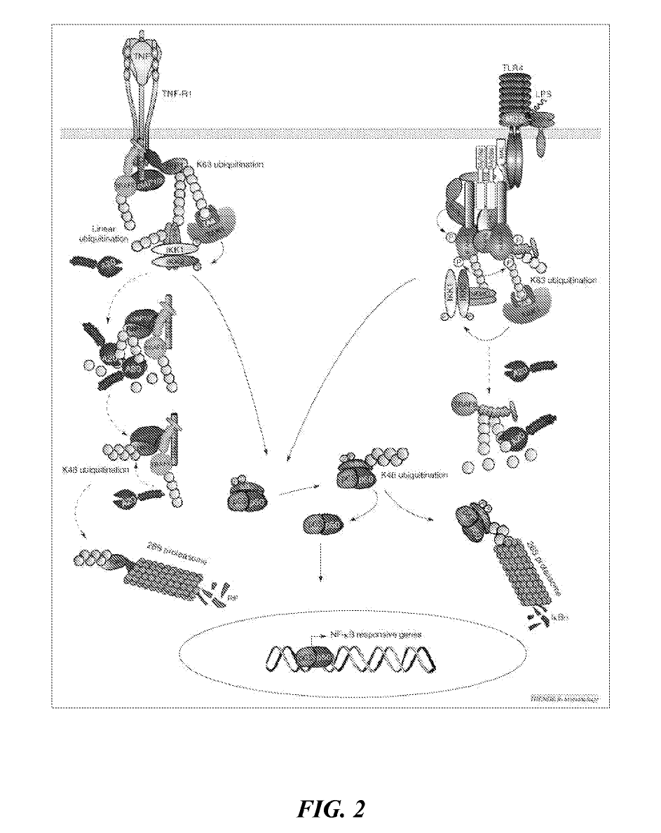

The gene TNFAIP3 encodes A20, a TNF-α-inducible zinc-finger protein that temporally limits immune responses by inhibiting NF-κB activation and terminating NF-κB mediated responses. TNIP3 encodes TNFAIP3 interacting protein 3, which interacts with A20 to inhibit NF-κB. See FIG. 2.

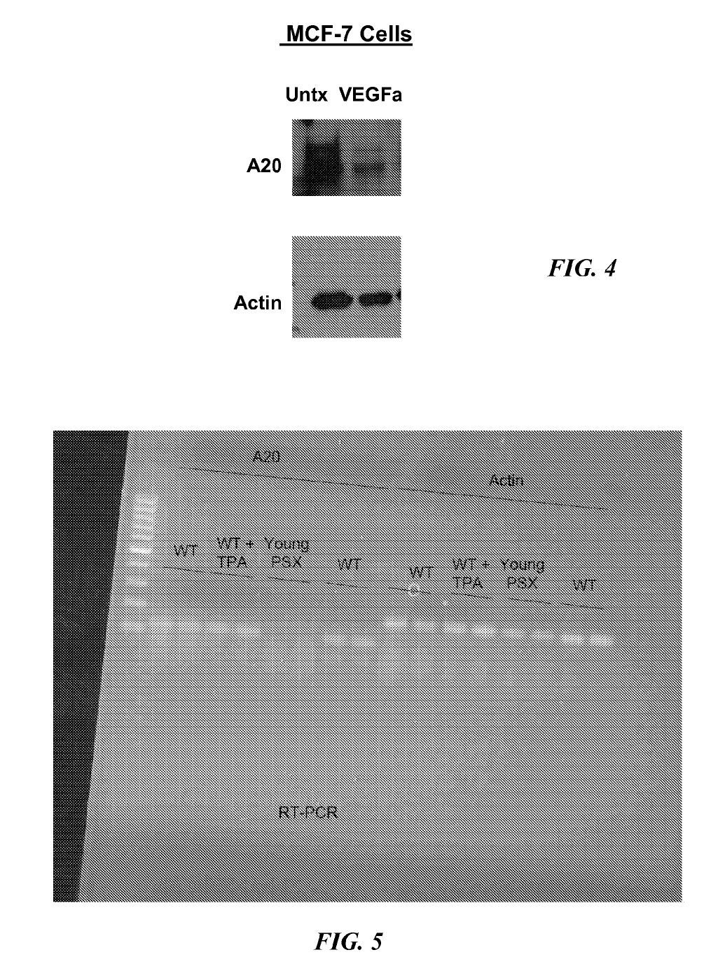

MCF-7 cell cultures over-expressing A20 were treated with VEGF-a. MCF-7 cells were obtained from ATCC (Manassas, Va.) and grown in culture per ATCC recommendations. Cells were passed the night before treatment and plated at 3×10⁵ cells in 6-well plates. 100 ng of human VEGF-a (Peprotech, #100-20) dissolved in DMEM was added to the treated cells for 1 hours. A20 is constitutively expressed in MCF-7 cells. As shown in FIG. 4, A20 is decreased in MCF-7 cells after treatment with VEGF-a. This demonstrates a possible mechanism by which VEGF-a overexpression in the K14-VEGFa mice leads to a decrease in A20 expression.

### Example 3

### Role of A20 in K14/VEGF Mouse Model

Using skin tissue samples from healthy control (WT) mice, WT (mice) treated with 12-O-tetradecanoyl phorbol-13-acetate (“TPA”), young 4 week old PSX mice, quantitative RT-PCR was performed to assess A20 and actin (control) expression levels. Mice were treated for 2 hours with topical TPA or vehice control. Mice were sacrificed and skin samples were placed in RNA Later (Qiagen). RNA was purified after tissue homogenization with the Qiagen RNA purification kit. RT-PCR was performed using primers for Actin (Forward: GCTGTGCTGTCCCTGTATGCCTCT, SEQ ID NO: 123; and Reverse: CCTCTCAGCTGTGGTGGTGAAGC, SEQ ID NO: 124) and A20 (Forward: AGCAAGTGCAGGAAAGCTGGCT, SEQ ID NO: 125; and Reverse: GCTTTCGCAGAGGCAGTAACAG, SEQ ID NO: 126) TPA is known to induce Tₕ17-like response in transgenic K14/VEGF mice (Hvid et al., “TPA induction leads to a Tₕ17-like Response in Transgenic K14/VEGF Mice: A Novel in vivo Screening Model of Psoriasis,” Int. Immunol. (2008) 20 (8): 1097-1106 (2008), which is hereby incorporated by reference in its entirety). FIG. 5 is a gel showing detection of PCR products following quantitative RT-PCR analysis of A20 and actin mRNA expression levels in wildtype (WT) mice, WT mice treated with 10 microliters TPA, and 4-week old (young) PSX mice.

In a separate quantitative RT-PCR analysis that also included 24-week old PSX mice, the results revealed that there was a 10-fold decrease in A20 transcripts in young PSX mice compared to young FVB controls. There was no significant difference in A20 transcript levels between older PSX mice and the controls.

Quantitative RT-PCR also was performed to assess whether any differences exist in the expression levels of the deubiquitinating enzyme CYLD and IκBα in 4-week old and 24-week old PSX mice compared to WT. No significant differences were identified for these other innate NF-κB inhibitors.

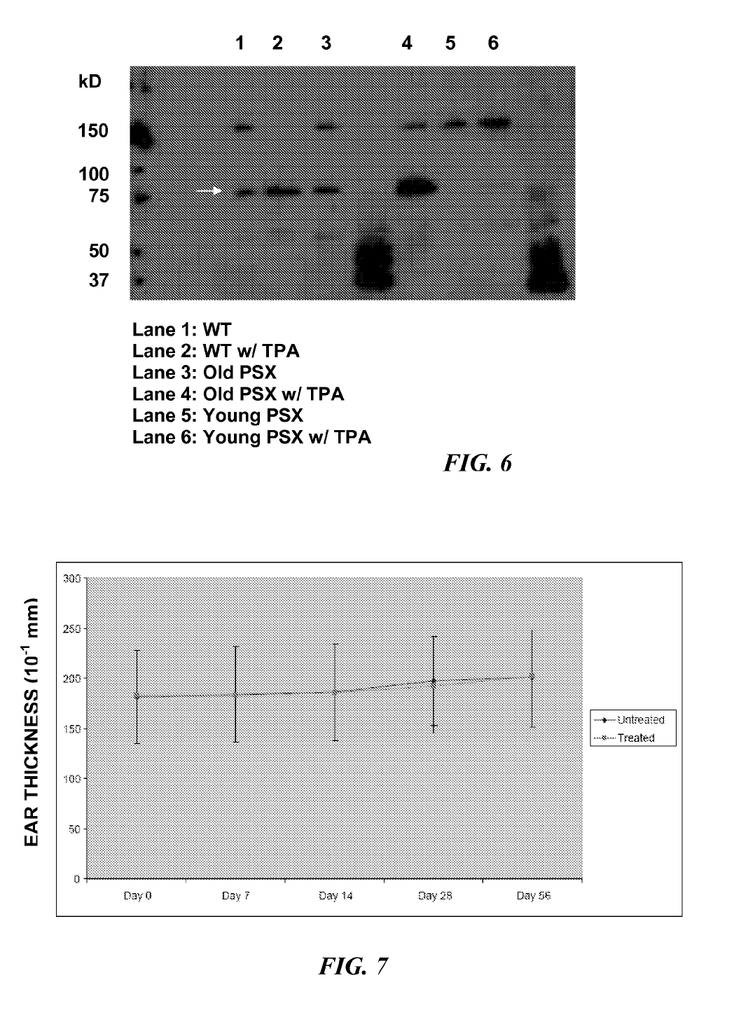

A Western Blot (FIG. 6) was performed on samples obtained from WT mice (lane 1), WT mice treated with TPA (lane 2), 4-week old (young) PSX mice without and with TPA treatment (lanes 3 and 4), and 24-week old (old) PSX mice without and with TPA treatment (lanes 5 and 6). An arrow indicates the band representing A20 running between markers 75 and 100 kD. These Western data confirm that A20 protein levels are significantly decreased in young PSX mice (with a predisposition for psoriasis) as compared to WT mice and older PSX mice. Moreover, because A20 is a gene induced by NF-κB activity and TPA is a potent activator of NF-κB, a robust induction is noted in the wild-type and old mice with developed psoriasis compared to the young, psoriasis-prone mice. This underlines why the NBD peptide is potent prior to psoriasis development or for treating early stage lesions, but not at later time points. The significant inflammation of the older psoriasis mice overcomes any deficit in A20, likely due to the many inflammatory mediators driving NF-κB activity. Whereas in uninflamed skin A20 plays a larger role in preventing NF-κB activity and thereby places a major role in preventing inflammation, the loss of A20 in the young PSX mice makes them prone to inflammation through NF-κB activity.

### Example 4

### Inhibition of Psoriatic Lesion Development in Juvenile PSX Mouse but Not Adult PSX Mouse

The NF-κB inhibitor used for the treatments in this example was the fusion protein drqikiwfqnrrmkwkkTALDWSWLQTE (SEQ ID NO: 122), where lower case letters indicate D-amino acid residues and upper case letters indicate L-amino acid residues. This inhibitor includes a translocation domain of Antennapedia and an Nemo Binding Domain (NBD) peptide.

NF-κB Inhibition with the pBD Peptide has been a subject of research in the art. See Kiessling et al , “Inhibition of Constitutive Activated Nuclear Factor-kappaB Induces Reactive Oxygen Species Species- and Iron-Dependent Cell Death in Cutaneous T-cell Lymphoma.,” Cancer Res. 69(6):2365-74 (2009); Ianaro et al., “NEMO-Binding Domain Peptide Inhibits Proliferation of Human Melanoma Cells,” Cancer Lett. 274(2):331-6 (2009); Shibata et al., “Cutting Edge: The IkappaB Kinase (IKK) Inhibitor, NEMO-Binding Domain Peptide, Blocks Inflammatory Injury in Murine Colitis,” J. Immunol. 179(5):2681-5 (2007); di Meglio et al., “Amelioration of Acute Inflammation by System Administration of a Cell-Permeable Peptide Inhibitor of NF-KappaB Activation,” Arthritis Rheum. 52(3):951-8 (2005); May et al., “Selective Inhibition of NF-kappaB Activation by a Peptide that Blocks the Interaction of NEMO With the IkappaB Kinase Complex,” Science 289(5484):1550-4 (2000), each of which is hereby incorporated by reference in its entirety.

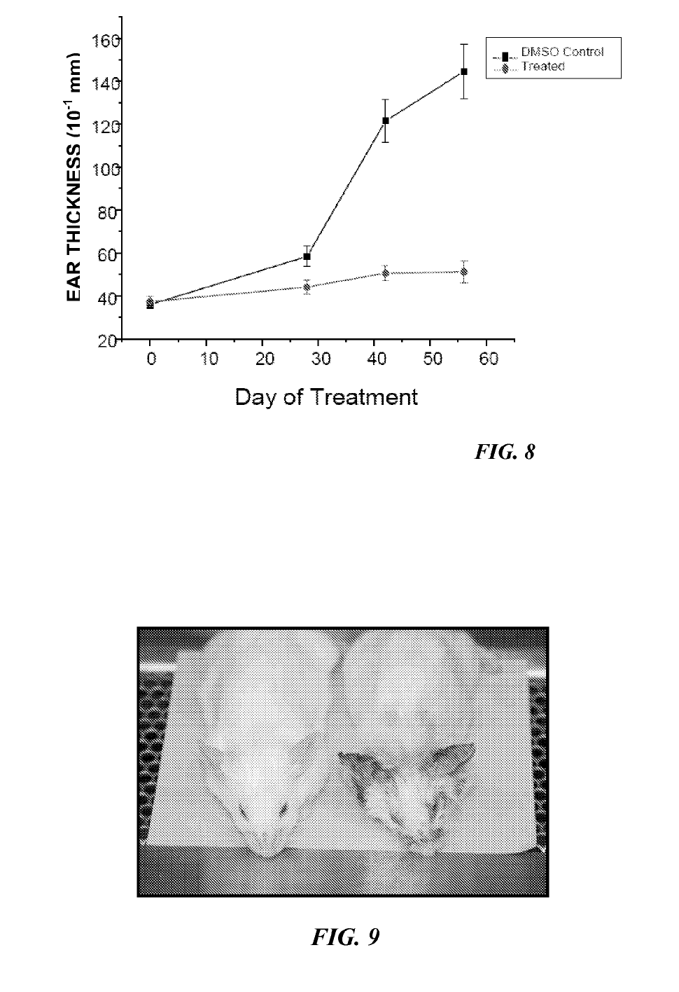

The peptide was formulated and administered as an intraperitoneal injection of 50 micrograms to either PSX mice after they had fully developed psoriatic skin lesions (FIG. 7) or prior to the development of psoriatic plaques when the ears were still uninvolved (FIG. 8). Assessment of the development of psoriatic lesions was measured by the thickness of ear tissue. As shown in FIG. 7, the treatment had no effect on fully developed psoriatic skin lesions. However, the treatment of PSX mice prior to the development of psoriatic plaques prevented the thickening of the ear and inhibited development of phenotypic lesions of psoriasis. The treatment quite clearly prevented the thickening of the ear and development of phenotypic lesions of psoriasis as seen in vehicle controls (FIG. 9).

### Example 5

### Topical Delivery of NBD Peptide

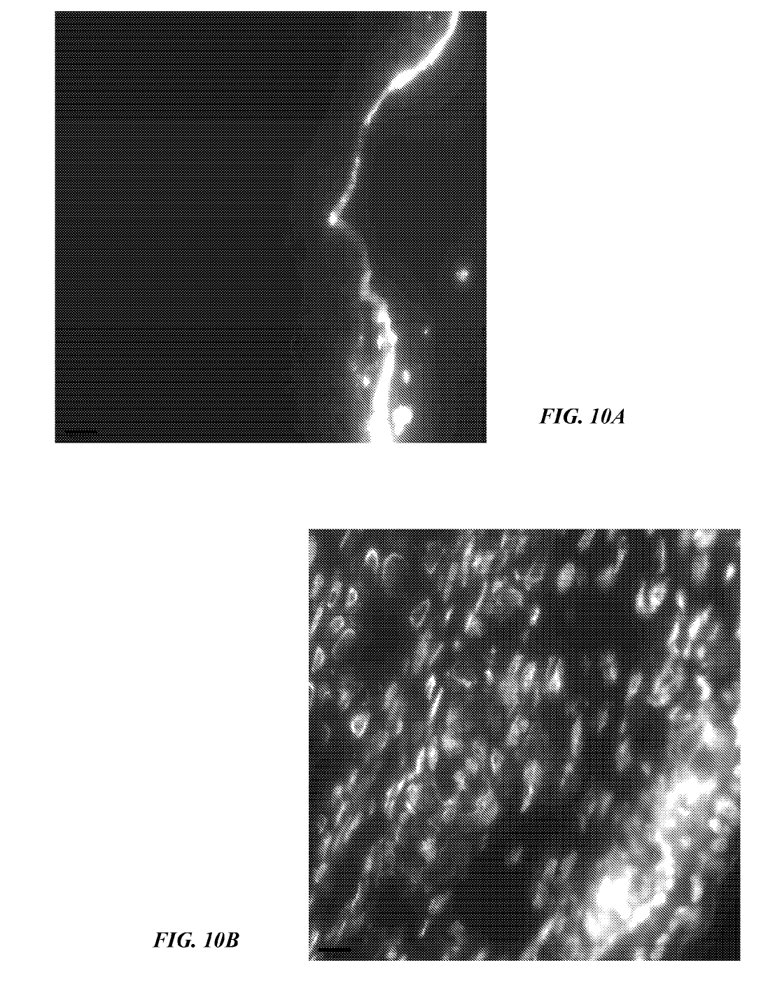

To assess the ability to deliver the NBD peptide of SEQ ID NO: 122 topically, the peptide was conjugated to the fluorophore FITC, and the labeled peptide prepared in two different formulations. The first formulation consisted of the labeled peptide dissolved in vaseline ointment, and the second formulation consisted of the labeled peptide dissolved in DMSO. In FIG. 10A, peptide is limited to the stratum corneum following administration of the peptide in vaseline ointment; the peptide was not absorbed into the skin. In FIG. 10B, peptide uptake is prevalent among the cells in both the epidermis and dermis following administration of the peptide in DMSO.

### Discussion of Examples 1-5

The importance of NF-κB in inflammation is well established. Most inflammatory cytokines activate NF-κB, and once activated NF-κB can upregulate proteins important for both the innate and adaptive immunity. Past studies have shown that patients with psoriasis have increased levels of NF-κB activity in lesional skin compared to unaffected patients. However, the role of NF-κB in the pathogenesis of psoriasis was relatively unknown.

Genetic studies have shown that a number of genetic polymorphisms, expressing proteins important in the NF-κB pathway, are associated with the susceptibility of psoriasis. Recently several proteins involved in tumor necrosis factor alpha (TNF-α) signaling to NF-κB, including TNFAIP3 and TNIP1, have been identified. Using a mouse model that is homozygous for the K14-VEGF transgene, this model was shown to develop psoriasiform lesions on the ears spontaneously and after trauma. Using this model, the innate NF-κB inhibitor A20 was identified as possibly being involved in the hyperactivity of NF-κB signaling in early disease, whereas the inhibitors CYLD and IκBα appear not be involved in early disease progression.

The results presented in the preceding examples demonstrate that inhibitors of NF-κB are surprisingly ineffective for treatment of advanced psoriatic lesions in this mouse model, indicating that NF-κB plays little or no role in the maintenance of the advanced stage inflammatory response. Rather, inhibitors of NF-κB are shown to be effective only for preventing the development of early stage psoriatic lesions or inhibiting the onset of psoriatic lesion formation. This indicates that there may be distinct signals for the initiation and maintenance of psoriatic plaques, with NF-κB contributing a crucial role only in the early phases of this disease. This discrepancy in NF-κB activity and, hence, the utility of NF-κB inhibitors as a treatment of only early stage psoriatic lesions is novel.

Further, it was demonstrated that NF-κB inhibitors can be delivered across the skin, allowing for topical application of formulations to sites on the body of the patient where psoriatic lesion formation normally occurs. Such treatments should prove effective to prevent onset of psoriatic lesions, or the treatment of early stage psoriatic lesions to prevent their further development.

It was also found that, independent of the mouse model, VEGF signaling causes a decrease in the innate NF-κB inhibitor A20, thereby creating a scenario of NF-κB hyperactivity. This is consistent with findings of increased NF-κB activity in human psoriatic plaques. It is possible that this dysregulated NF-κB signaling contributes to the early phases of psoriatic plaque development through upregulation of certain inflammatory mediators. Further understanding of the signals which contribute to the early and late phases of psoriatic plaque development may provide key therapeutic targets in future treatment of this disease. For instance, treatment with agents that upregulate A20 expression, including gene therapy approaches for A20 overexpression, should prove useful in combination with inhibitors of NF-κB.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of inhibiting onset of or preventing development of a psoriatic lesion in a patient having psoriasis, the method comprising:
administering to a patient having psoriasis an effective amount of an agent that inhibits NF-κB activity under conditions effective to inhibit onset of or prevent development of psoriatic lesions.

2. A method of treating an early stage psoriatic lesion on a patient comprising:
contacting the early stage psoriatic lesion of a patient with an effective amount of an agent that inhibits NF-κB activity, whereby said contacting inhibits development of the early stage psoriatic lesion.

3. The method according to claim 1, wherein the patient is asymptomatic at the time of administering.

4. The method according to claim 1, wherein the patient has one or more lesions of less than 25 mm² in size.

5. The method according to claim 1, wherein the patient has only lesions of less than 25 mm² in size.

6. The method according to claim 1, wherein said administering is carried out systemically.

7. The method according to claim 1, wherein said administering is carried out topically.

8. The method according to claim 1, wherein the patient is characterized by a low expression level of A20.

9. The method according to claim 1, wherein the agent that inhibits NF-κB activity is a fusion polypeptide comprising a membrane translocation domain and a NEMO binding sequence.

10. The method according to claim 9, wherein the membrane translocation peptide comprises an antennapedia homeodomain third helix polypeptide, an HIV-1 Tat polypeptide, or a peptide comprising 6-15 amino acid residues, where at least five of the 6-15 amino acid residues are basic amino acid residues independently selected from L-arginine, D-arginine, L-lysine and D-lysine.

11. The method according to claim 10, wherein the membrane translocation peptide comprises the amino acid sequence of RRMKWKK (SEQ ID NO: 96); YGRKKRRQRRR (SEQ ID NO: 97); ygrkkrrqrrr (SEQ ID NO: 98); YARKARRQARR (SEQ ID NO: 99); yarkarrqarr (SEQ ID NO: 100); YARAARRAARR (SEQ ID NO: 101); yaraarraarr (SEQ ID NO: 102); rrmkwkk (SEQ ID NO: 103); or poly-L-Arg or poly-D-Arg comprising 6 to 11 Arg residues;
wherein lower case letters indicate D-amino acid residues and upper case letters indicate L-amino acid residues).

12. The method according to claim 9, wherein the NEMO binding peptide is selected from the group consisting of TALDWSWLQTE (SEQ ID NO: 1); LDWSWLQTE (SEQ ID NO: 2); TALDWSWL (SEQ ID NO: 3); ALDWSWLQTE (SEQ ID NO: 4); LDWSWLQTE (SEQ ID NO: 5); LDWSWL (SEQ ID NO: 6); TALDWSWLQT (SEQ ID NO: 7); TALDWSWLQ (SEQ ID NO: 8); ALDWSWLQT (SEQ ID NO: 9); LDWSWLQ (SEQ ID NO: 10); LDWSWLQT (SEQ ID NO: 11); ADWSWL (SEQ ID NO: 12); LDWSWA (SEQ ID NO: 13); ADWSWA (SEQ ID NO: 14); LDFSWL (SEQ ID NO: 15); LDYSWL (SEQ ID NO: 16); LDWAWL (SEQ ID NO: 17); LDWEWL (SEQ ID NO: 18); TAADWSWLQTE (SEQ ID NO: 19); ADWSWLQTE (SEQ ID NO: 20); TAADWSWL (SEQ ID NO: 21); AADWSWLQTE (SEQ ID NO: 22); ADWSWLQTE (SEQ ID NO: 23); ADWSWL (SEQ ID NO: 24); TAADWSWLQT (SEQ ID NO: 25); TAADWSWLQ (SEQ ID NO: 26); AADWSWLQT (SEQ ID NO: 27); ADWSWLQ (SEQ ID NO: 28); ADWSWLQT (SEQ ID NO: 29); ALDWSWAQTE (SEQ ID NO: 30); LDWSWAQTE (SEQ ID NO: 31); TALDWSWA (SEQ ID NO: 32); ALDWSWAQTE (SEQ ID NO: 33); LDWSWAQTE (SEQ ID NO: 34); LDWSWA (SEQ ID NO: 35); TALDWSWAQT (SEQ ID NO: 36); TALDWSWAQ (SEQ ID NO: 37); ALDWSWAQT (SEQ ID NO: 38); LDWSWAQ (SEQ ID NO: 39); LDWSWAQT (SEQ ID NO: 40); TAADWSWAQTE (SEQ ID NO: 41); ADWSWAQTE (SEQ ID NO: 42); TAADWSWA (SEQ ID NO: 43); AADWSWAQTE (SEQ ID NO: 44); ADWSWAQTE (SEQ ID NO: 45); ADWSWA (SEQ ID NO: 46); TAADWSWAQT (SEQ ID NO: 47; TAADWSWAQ (SEQ ID NO: 48); AADWSWAQT (SEQ ID NO: 49); ADWSWAQ (SEQ ID NO: 50); ADWSWAQT (SEQ ID NO: 51); TALDFSWLQTE (SEQ ID NO: 52); LDFSWLQTE (SEQ ID NO: 53); TALDFSWL (SEQ ID NO: 54); ALDFSWLQTE (SEQ ID NO: 55); LDFSWLQTE (SEQ ID NO: 56); LDFSWL (SEQ ID NO: 57); TALDFSWLQT (SEQ ID NO: 58); TALDFSWLQ (SEQ ID NO: 59); ALDFSWLQT (SEQ ID NO: 60); LDFSWLQ (SEQ ID NO: 61); LDFSWLQT (SEQ ID NO: 62); TALDYSWLQTE (SEQ ID NO: 63); LDYSWLQTE (SEQ ID NO: 64); TALDYSWL (SEQ ID NO: 65); ALDYSWLQTE (SEQ ID NO: 66); LDYSWLQTE (SEQ ID NO: 67); LDYSWL (SEQ ID NO: 68); TALDYSWLQT (SEQ ID NO: 69); TALDYSWLQ (SEQ ID NO: 70); ALDYSWLQT (SEQ ID NO: 71); LDYSWLQ (SEQ ID NO: 72); LDYSWLQT (SEQ ID NO: 73); TALDWAWLQTE (SEQ ID NO: 74); LDWAWLQTE (SEQ ID NO: 75); TALDWAWL (SEQ ID NO: 76); ALDWAWLQTE (SEQ ID NO: 77); LDWAWLQTE (SEQ ID NO: 78); LDWAWL (SEQ ID NO: 79); TALDWAWLQT (SEQ ID NO: 80); TALDWAWLQ (SEQ ID NO: 81); ALDWAWLQT (SEQ ID NO: 82); LDWAWLQ (SEQ ID NO: 83); LDWAWLQT (SEQ ID NO: 84); TALDWEWLQTE (SEQ ID NO: 85); LDWEWLQTE (SEQ ID NO: 86); TALDWEWL (SEQ ID NO: 87); ALDWEWLQTE (SEQ ID NO: 88); LDWEWLQTE (SEQ ID NO: 89); LDWEWL (SEQ ID NO: 90); TALDWEWLQT (SEQ ID NO: 91); TALDWEWLQ (SEQ ID NO: 92); ALDWEWLQT (SEQ ID NO: 93); LDWEWLQ (SEQ ID NO: 94); and LDWEWLQT (SEQ ID NO: 95).

13. The method according to claim 9, wherein the agent comprises drqikiwfqnrrmkwkkTALDWSWLQTE (SEQ ID NO: 122), RRMKWKKTALDWSWLQTE (SEQ ID NO: 104); rrmkwkkTALDWSWLQTE (SEQ ID NO: 105); YGRKKRRQRRRTALDWSWLQTE (SEQ ID NO: 106); ygrkkrrqrrrTALDWSWLQTE (SEQ ID NO: 107); rrrrrrrTALDWSWLQTE (SEQ ID NO: 108); RRRRRRRTALDWSWLQTE (SEQ ID NO: 109); YARKARRQARRTALDWSWLQTE (SEQ ID NO: 110); yarkarrqarrTALDWSWLQTE (SEQ ID NO: 111); YARAARRAARRTALDWSWLQTE (SEQ ID NO: 112); yaraarraarrTALDWSWLQTE (SEQ ID NO: 113); YGRKKRRQRRRLDWSWL (SEQ ID NO: 114); ygrkkrrqrrrLDWSWL (SEQ ID NO: 115); RRMKWKKLDWSWL (SEQ ID NO: 116); rrmnkwkkLDWSWL (SEQ ID NO: 117); rrrrrrrLDWSWL (SEQ ID NO: 118); YARAARRAARRLDWSWL (SEQ ID NO: 119); yaraarraarrLDWSWL (SEQ ID NO: 120); or RRRRRRRLDWSWL (SEQ ID NO: 121),
wherein lower case letters indicate D-amino acid residues and upper case letters indicate L-amino acid residues.

14. The method according to claim 1, wherein the agent is an inhibitor of IKK.

15. The method according to claim 14, wherein the IKK inhibitor is PS1145, PS341, thalidomide, bortezomib, herbimycin A, sodium salicylate, a retinoid-related compound, a cyclopentenone prostaglandin, vinpocetine, an anilinopyrimidine derivatives, an indole or benzimidazole derivative, a 4-aryl pyridine derivative, BMS-345541, SC-514, or TPCA-1.

16. The method according to claim 1 further comprising:
administering to the patient or contacting the early stage lesion with one or more additional therapeutic agents.

17. The method according to claim 16, wherein the one or more additional therapeutic agents is selected from the group of corticosteroids, TNF-α inhibitors, vitamin D analogs, retinoids, calcineurin inhibitors, phototherapy, methotrexate, cyclosporine, hydroxyurea, and thioguanine.

18. The method according to claim 16, wherein said administering the agent that inhibits NF-κB activity is carried out after beginning a course of said administering the one or more additional therapeutic agents.

19. The method according to claim 16, wherein said administering the agent that inhibits NF-κB activity is carried out after ending a course of said administering the one or more additional therapeutic agents.

20. The method according to claim 16, wherein said administering the agent that inhibits NF-κB activity is carried out concurrently with said administering the one or more additional therapeutic agents.

21. The method according to claim 1, wherein the agent that inhibits NF-κB activity is a transgene encoding A20.

22. A method of treating a patient having an inflammatory condition, the method comprising:
administering to a patient having an inflammatory condition an effective amount of a transgene encoding A20, whereby expression of the transgene inhibits NF-κB activity and is effective to treat the patient for the inflammatory condition.

23. The method according to claim 22, wherein the transgene is present in an infective delivery vehicle.

24. The method according to claim 22, wherein the transgene is present as naked DNA in a composition suitable for said administration.


SEQUENCE LISTING (126 sequences)

SEQ ID NO: 1 - protein, 11 aa, Artificial Sequence
  NEMO Binding Sequence
TALDWSWLQTE

SEQ ID NO: 2 - protein, 9 aa, Artificial Sequence
  NEMO Binding Sequence
LDWSWLQTE

SEQ ID NO: 3 - protein, 8 aa, Artificial Sequence
  NEMO Binding Sequence
TALDWSWL

SEQ ID NO: 4 - protein, 10 aa, Artificial Sequence
  NEMO Binding Sequence
ALDWSWLQTE

SEQ ID NO: 5 - protein, 9 aa, Artificial Sequence
  NEMO Binding Sequence
LDWSWLQTE

SEQ ID NO: 6 - protein, 6 aa, Artificial Sequence
  NEMO Binding Sequence
LDWSWL

SEQ ID NO: 7 - protein, 10 aa, Artificial Sequence
  NEMO Binding Sequence
TALDWSWLQT

SEQ ID NO: 8 - protein, 9 aa, Artificial Sequence
  NEMO Binding Sequence
TALDWSWLQ

SEQ ID NO: 9 - protein, 9 aa, Artificial Sequence
  NEMO Binding Sequence
ALDWSWLQT

SEQ ID NO: 10 - protein, 7 aa, Artificial Sequence
  NEMO Binding Sequence
LDWSWLQ

SEQ ID NO: 11 - protein, 8 aa, Artificial Sequence
  NEMO Binding Sequence
LDWSWLQT

SEQ ID NO: 12 - protein, 6 aa, Artificial Sequence
  NEMO Binding Sequence
ADWSWL

SEQ ID NO: 13 - protein, 6 aa, Artificial Sequence
  NEMO Binding Sequence
LDWSWA

SEQ ID NO: 14 - protein, 6 aa, Artificial Sequence
  NEMO Binding Sequence
ADWSWA

SEQ ID NO: 15 - protein, 6 aa, Artificial Sequence
  NEMO Binding Sequence
LDFSWL

SEQ ID NO: 16 - protein, 6 aa, Artificial Sequence
  NEMO Binding Sequence
LDYSWL

SEQ ID NO: 17 - protein, 6 aa, Artificial Sequence
  NEMO Binding Sequence
LDWAWL

SEQ ID NO: 18 - protein, 6 aa, Artificial Sequence
  NEMO Binding Sequence
LDWEWL

SEQ ID NO: 19 - protein, 11 aa, Artificial Sequence
  NEMO Binding Sequence
TAADWSWLQTE

SEQ ID NO: 20 - protein, 9 aa, Artificial Sequence
  NEMO Binding Sequence
ADWSWLQTE

SEQ ID NO: 21 - protein, 8 aa, Artificial Sequence
  NEMO Binding Sequence
TAADWSWL

SEQ ID NO: 22 - protein, 10 aa, Artificial Sequence
  NEMO Binding Sequence
AADWSWLQTE

SEQ ID NO: 23 - protein, 9 aa, Artificial Sequence
  NEMO Binding Sequence
ADWSWLQTE

SEQ ID NO: 24 - protein, 6 aa, Artificial Sequence
  NEMO Binding Sequence
ADWSWL

SEQ ID NO: 25 - protein, 10 aa, Artificial Sequence
  NEMO Binding Sequence
TAADWSWLQT

SEQ ID NO: 26 - protein, 9 aa, Artificial Sequence
  NEMO Binding Sequence
TAADWSWLQ

SEQ ID NO: 27 - protein, 9 aa, Artificial Sequence
  NEMO Binding Sequence
AADWSWLQT

SEQ ID NO: 28 - protein, 7 aa, Artificial Sequence
  NEMO Binding Sequence
ADWSWLQ

SEQ ID NO: 29 - protein, 8 aa, Artificial Sequence
  NEMO Binding Sequence
ADWSWLQT

SEQ ID NO: 30 - protein, 10 aa, Artificial Sequence
  NEMO Binding Sequence
ALDWSWAQTE

SEQ ID NO: 31 - protein, 9 aa, Artificial Sequence
  NEMO Binding Sequence
LDWSWAQTE

SEQ ID NO: 32 - protein, 8 aa, Artificial Sequence
  NEMO Binding Sequence
TALDWSWA

SEQ ID NO: 33 - protein, 10 aa, Artificial Sequence
  NEMO Binding Sequence
ALDWSWAQTE

SEQ ID NO: 34 - protein, 9 aa, Artificial Sequence
  NEMO Binding Sequence
LDWSWAQTE

SEQ ID NO: 35 - protein, 6 aa, Artificial Sequence
  NEMO Binding Sequence
LDWSWA

SEQ ID NO: 36 - protein, 10 aa, Artificial Sequence
  NEMO Binding Sequence
TALDWSWAQT

SEQ ID NO: 37 - protein, 9 aa, Artificial Sequence
  NEMO Binding Sequence
TALDWSWAQ

SEQ ID NO: 38 - protein, 9 aa, Artificial Sequence
  NEMO Binding Sequence
ALDWSWAQT

SEQ ID NO: 39 - protein, 7 aa, Artificial Sequence
  NEMO Binding Sequence
LDWSWAQ

SEQ ID NO: 40 - protein, 8 aa, Artificial Sequence
  NEMO Binding Sequence
LDWSWAQT

SEQ ID NO: 41 - protein, 11 aa, Artificial Sequence
  NEMO Binding Sequence
TAADWSWAQTE

SEQ ID NO: 42 - protein, 9 aa, Artificial Sequence
  NEMO Binding Sequence
ADWSWAQTE

SEQ ID NO: 43 - protein, 8 aa, Artificial Sequence
  NEMO Binding Sequence
TAADWSWA

SEQ ID NO: 44 - protein, 10 aa, Artificial Sequence
  NEMO Binding Sequence
AADWSWAQTE

SEQ ID NO: 45 - protein, 9 aa, Artificial Sequence
  NEMO Binding Sequence
ADWSWAQTE

SEQ ID NO: 46 - protein, 6 aa, Artificial Sequence
  NEMO Binding Sequence
ADWSWA

SEQ ID NO: 47 - protein, 10 aa, Artificial Sequence
  NEMO Binding Sequence
TAADWSWAQT

SEQ ID NO: 48 - protein, 9 aa, Artificial Sequence
  NEMO Binding Sequence
TAADWSWAQ

SEQ ID NO: 49 - protein, 9 aa, Artificial Sequence
  NEMO Binding Sequence
AADWSWAQT

SEQ ID NO: 50 - protein, 7 aa, Artificial Sequence
  NEMO Binding Sequence
ADWSWAQ

SEQ ID NO: 51 - protein, 8 aa, Artificial Sequence
  NEMO Binding Sequence
ADWSWAQT

SEQ ID NO: 52 - protein, 11 aa, Artificial Sequence
  NEMO Binding Sequence
TALDFSWLQTE

SEQ ID NO: 53 - protein, 9 aa, Artificial Sequence
  NEMO Binding Sequence
LDFSWLQTE

SEQ ID NO: 54 - protein, 8 aa, Artificial Sequence
  NEMO Binding Sequence
TALDFSWL

SEQ ID NO: 55 - protein, 10 aa, Artificial Sequence
  NEMO Binding Sequence
ALDFSWLQTE

SEQ ID NO: 56 - protein, 9 aa, Artificial Sequence
  NEMO Binding Sequence
LDFSWLQTE

SEQ ID NO: 57 - protein, 6 aa, Artificial Sequence
  NEMO Binding Sequence
LDFSWL

SEQ ID NO: 58 - protein, 10 aa, Artificial Sequence
  NEMO Binding Sequence
TALDFSWLQT

SEQ ID NO: 59 - protein, 9 aa, Artificial Sequence
  NEMO Binding Sequence
TALDFSWLQ

SEQ ID NO: 60 - protein, 9 aa, Artificial Sequence
  NEMO Binding Sequence
ALDFSWLQT

SEQ ID NO: 61 - protein, 7 aa, Artificial Sequence
  NEMO Binding Sequence
LDFSWLQ

SEQ ID NO: 62 - protein, 8 aa, Artificial Sequence
  NEMO Binding Sequence
LDFSWLQT

SEQ ID NO: 63 - protein, 11 aa, Artificial Sequence
  NEMO Binding Sequence
TALDYSWLQTE

SEQ ID NO: 64 - protein, 9 aa, Artificial Sequence
  NEMO Binding Sequence
LDYSWLQTE

SEQ ID NO: 65 - protein, 8 aa, Artificial Sequence
  NEMO Binding Sequence
TALDYSWL

SEQ ID NO: 66 - protein, 10 aa, Artificial Sequence
  NEMO Binding Sequence
ALDYSWLQTE

SEQ ID NO: 67 - protein, 9 aa, Artificial Sequence
  NEMO Binding Sequence
LDYSWLQTE

SEQ ID NO: 68 - protein, 6 aa, Artificial Sequence
  NEMO Binding Sequence
LDYSWL

SEQ ID NO: 69 - protein, 10 aa, Artificial Sequence
  NEMO Binding Sequence
TALDYSWLQT

SEQ ID NO: 70 - protein, 9 aa, Artificial Sequence
  NEMO Binding Sequence
TALDYSWLQ

SEQ ID NO: 71 - protein, 9 aa, Artificial Sequence
  NEMO Binding Sequence
ALDYSWLQT

SEQ ID NO: 72 - protein, 7 aa, Artificial Sequence
  NEMO Binding Sequence
LDYSWLQ

SEQ ID NO: 73 - protein, 8 aa, Artificial Sequence
  NEMO Binding Sequence
LDYSWLQT

SEQ ID NO: 74 - protein, 11 aa, Artificial Sequence
  NEMO Binding Sequence
TALDWAWLQTE

SEQ ID NO: 75 - protein, 9 aa, Artificial Sequence
  NEMO Binding Sequence
LDWAWLQTE

SEQ ID NO: 76 - protein, 8 aa, Artificial Sequence
  NEMO Binding Sequence
TALDWAWL

SEQ ID NO: 77 - protein, 10 aa, Artificial Sequence
  NEMO Binding Sequence
ALDWAWLQTE

SEQ ID NO: 78 - protein, 9 aa, Artificial Sequence
  NEMO Binding Sequence
LDWAWLQTE

SEQ ID NO: 79 - protein, 6 aa, Artificial Sequence
  NEMO Binding Sequence
LDWAWL

SEQ ID NO: 80 - protein, 10 aa, Artificial Sequence
  NEMO Binding Sequence
TALDWAWLQT

SEQ ID NO: 81 - protein, 9 aa, Artificial Sequence
  NEMO Binding Sequence
TALDWAWLQ

SEQ ID NO: 82 - protein, 9 aa, Artificial Sequence
  NEMO Binding Sequence
ALDWAWLQT

SEQ ID NO: 83 - protein, 7 aa, Artificial Sequence
  NEMO Binding Sequence
LDWAWLQ

SEQ ID NO: 84 - protein, 8 aa, Artificial Sequence
  NEMO Binding Sequence
LDWAWLQT

SEQ ID NO: 85 - protein, 11 aa, Artificial Sequence
  NEMO Binding Sequence
TALDWEWLQTE

SEQ ID NO: 86 - protein, 9 aa, Artificial Sequence
  NEMO Binding Sequence
LDWEWLQTE

SEQ ID NO: 87 - protein, 8 aa, Artificial Sequence
  NEMO Binding Sequence
TALDWEWL

SEQ ID NO: 88 - protein, 10 aa, Artificial Sequence
  NEMO Binding Sequence
ALDWEWLQTE

SEQ ID NO: 89 - protein, 9 aa, Artificial Sequence
  NEMO Binding Sequence
LDWEWLQTE

SEQ ID NO: 90 - protein, 6 aa, Artificial Sequence
  NEMO Binding Sequence
LDWEWL

SEQ ID NO: 91 - protein, 10 aa, Artificial Sequence
  NEMO Binding Sequence
TALDWEWLQT

SEQ ID NO: 92 - protein, 9 aa, Artificial Sequence
  NEMO Binding Sequence
TALDWEWLQ

SEQ ID NO: 93 - protein, 9 aa, Artificial Sequence
  NEMO Binding Sequence
ALDWEWLQT

SEQ ID NO: 94 - protein, 7 aa, Artificial Sequence
  NEMO Binding Sequence
LDWEWLQ

SEQ ID NO: 95 - protein, 8 aa, Artificial Sequence
  NEMO Binding Sequence
LDWEWLQT

SEQ ID NO: 96 - protein, 7 aa, Artificial Sequence
  Membrane Translocation Domain
RRMKWKK

SEQ ID NO: 97 - protein, 11 aa, Artificial Sequence
  Membrane Translocation Domain
YGRKKRRQRRR

SEQ ID NO: 98 - protein, 11 aa, Artificial Sequence
  Membrane Translocation Domain
YGRKKRRQRRR

SEQ ID NO: 99 - protein, 11 aa, Artificial Sequence
  Membrane Translocation Domain
YARKARRQARR

SEQ ID NO: 100 - protein, 11 aa, Artificial Sequence
  Membrane Translocation Domain
YARKARRQARR

SEQ ID NO: 101 - protein, 11 aa, Artificial Sequence
  Membrane Translocation Domain
YARAARRAARR

SEQ ID NO: 102 - protein, 11 aa, Artificial Sequence
  Membrane Translocation Domain
YARAARRAARR

SEQ ID NO: 103 - protein, 7 aa, Artificial Sequence
  Membrane Translocation Domain
RRMKWKK

SEQ ID NO: 104 - protein, 18 aa, Artificial Sequence
  Anti-Inflammatory Compound
RRMKWKKTALDWSWLQTE

SEQ ID NO: 105 - protein, 18 aa, Artificial Sequence
  Anti-Inflammatory Compound
RRMKWKKTALDWSWLQTE

SEQ ID NO: 106 - protein, 22 aa, Artificial Sequence
  Anti-Inflammatory Compound
YGRKKRRQRRRTALDWSWLQTE

SEQ ID NO: 107 - protein, 22 aa, Artificial Sequence
  Anti-Inflammatory Compound
YGRKKRRQRRRTALDWSWLQTE

SEQ ID NO: 108 - protein, 18 aa, Artificial Sequence
  Anti-Inflammatory Compound
RRRRRRRTALDWSWLQTE

SEQ ID NO: 109 - protein, 18 aa, Artificial Sequence
  Anti-Inflammatory Compound
RRRRRRRTALDWSWLQTE

SEQ ID NO: 110 - protein, 22 aa, Artificial Sequence
  Anti-Inflammatory Compound
YARKARRQARRTALDWSWLQTE

SEQ ID NO: 111 - protein, 22 aa, Artificial Sequence
  Anti-Inflammatory Compound
YARKARRQARRTALDWSWLQTE

SEQ ID NO: 112 - protein, 22 aa, Artificial Sequence
  Anti-Inflammatory Compound
YARAARRAARRTALDWSWLQTE

SEQ ID NO: 113 - protein, 22 aa, Artificial Sequence
  Anti-Inflammatory Compound
YARAARRAARRTALDWSWLQTE

SEQ ID NO: 114 - protein, 17 aa, Artificial Sequence
  Anti-Inflammatory Compound
YGRKKRRQRRRLDWSWL

SEQ ID NO: 115 - protein, 17 aa, Artificial Sequence
  Anti-Inflammatory Compound
YGRKKRRQRRRLDWSWL

SEQ ID NO: 116 - protein, 13 aa, Artificial Sequence
  Anti-Inflammatory Compound
RRMKWKKLDWSWL

SEQ ID NO: 117 - protein, 14 aa, Artificial Sequence
  Anti-Inflammatory Compound
RRMNKWKKLDWSWL

SEQ ID NO: 118 - protein, 13 aa, Artificial Sequence
  Anti-Inflammatory Compound
RRRRRRRLDWSWL

SEQ ID NO: 119 - protein, 17 aa, Artificial Sequence
  Anti-Inflammatory Compound
YARAARRAARRLDWSWL

SEQ ID NO: 120 - protein, 17 aa, Artificial Sequence
  Anti-Inflammatory Compound
YARAARRAARRLDWSWL

SEQ ID NO: 121 - protein, 13 aa, Artificial Sequence
  Anti-Inflammatory Compound
RRRRRRRLDWSWL

SEQ ID NO: 122 - protein, 28 aa, Artificial Sequence
  Anti-Inflammatory Compound
DRQIKIWFQNRRMKWKKTALDWSWLQTE

SEQ ID NO: 123 - DNA, 24 bp, Artificial Sequence
  Actin Forward Primer
gctgtgctgtccctgtatgcctct

SEQ ID NO: 124 - DNA, 23 bp, Artificial Sequence
  Actin Reverse Primer
cctctcagctgtggtggtgaagc

SEQ ID NO: 125 - DNA, 22 bp, Artificial Sequence
  A20 Forward Primer
agcaagtgcaggaaagctggct

SEQ ID NO: 126 - DNA, 22 bp, Artificial Sequence
  A20 Reverse Primer
gctttcgcagaggcagtaacag